(19) United States
(12) Patent Application Publication
(10) Pub. No.: US 2018/0344865 A1
(43) Pub. Date: Dec. 6, 2018

(54) Methods of Increasing Fertility in a Female Subject

(71) Applicant: Palo Alto Investors, Palo Alto, CA (US)

(72) Inventors: Anthony Joonkyoo Yun, Menlo Park, CA (US); Conrad Minkyoo Yun, San Mateo, CA (US); Kimberly A. Bazar, Palo Alto, CA (US)

(21) Appl. No.: 15/976,416

(22) Filed: May 10, 2018

Related U.S. Application Data
(60) Provisional application No. 62/513,295, filed on May 31, 2017.

(51) Int. Cl. A61K 47/68; A61P 15/08

(52) U.S. Cl. CPC A61K 47/6803; A61K 2039/572; A61K 47/6849; A61P 15/08

(57) ABSTRACT

Methods of increasing fertility in a female subject are provided. Aspects of the methods include ablating senescent cells present in the reproductive system of the subject to increase the fertility of the female subject. Ablating senescent cells in the provided methods may include where senescent cells of the reproductive system are specifically ablated.

### CROSS-REFERENCE TO RELATED APPLICATIONS

Pursuant to 35 U.S.C. § 119 (e), this application claims priority to the filing date of U.S. Provisional Patent Application Ser. No. 62/513,295 filed May 31, 2017; the disclosure of which application is herein incorporated by reference.

### INTRODUCTION

In 2010, an estimated 48.5 million couples worldwide were unable to have a child after five years of trying (See Mascarenhas et al., PLoS Med. 2012; 9(12):e1001356). Global infertility prevalence rates are difficult to determine, due to the presence of both male and female factors which complicate making any estimate which specifically addressed the female contribution to a couple's infertility. However, studies have shown that more women in developed countries, the United States included, are waiting until later in life to start having children. In 2013, the U.S. saw a record high average age of 26 years old for women having their first child, an increase of 3.3 years since 1980 (Marin et al., National Vital Statistics Report. (2015) 64(1):1-66). The average age of first-time mothers is increasing because more women are waiting until their 30s and 40s to start having kids and fewer women are having their first children in their teens and 20s. Fertility treatments have facilitated this trend making it easier in some cases, and possible in others, for women to have children later in life.

Roughly 1.3 million patients receive infertility advice or treatment each year in the U.S. The emotional effects of infertility, whether a result of a medical condition preventing pregnancy or decreased fertility due to advanced age, negatively impact many relationships and marriages. One study of 200 couples seen consecutively at a fertility clinic, for example, found that half of the women and 15% of the men said that infertility was the most upsetting experience of their lives (see Harvard Mental Health Letter, May 2009). In addition, advanced infertility treatments are expensive. The average cost of in vitro fertilization treatment in the U.S. is currently about $11,000 to $12,000 and even less aggressive treatments such as intrauterine insemination and ovarian stimulation can cost many hundreds to a few thousand dollars. There are no indications that rates of infertility are dropping or that this trend increasing average age of first pregnancy will reverse.

### SUMMARY

Methods of increasing fertility in a female subject are provided. Aspects of the methods include ablating senescent cells present in the reproductive system of the subject to increase the fertility of the female subject. Ablating senescent cells in the provided methods may include where senescent cells of the reproductive system are specifically ablated.

### Definitions

The term “fertility”, as used herein, generally refers to the capacity to conceive or to induce conception of offspring. Accordingly, “infertility” generally refers to the inability to conceive or to induce conception of offspring. Infertility may be determined based on a duration of time, during which a mating couple or sexual partners having intercourse without contraception are unable to conceive offspring. In some instances, infertility may be indicated after a duration of about one year without conception. Female fertility may refer to the ability of a female to produce productive eggs or ova and/or the ability of a female to ovulate, i.e., discharge ova from the ovary. In some instances, infertility may be a result of menopause.

The term “menopause”, as used herein, generally refers to the end of menstruation of a female subject. Menopause includes a decline in estrogen resulting in a wide range of changes in various tissues that respond to estrogen, such as e.g., vagina, vulva, uterus, bladder, urethra, breasts, bones, heart, blood vessels, brain, skin, hair, mucous membranes, and the like. The most common symptom of menopause is a change in the menstrual cycle, but other symptoms may occur, including but not limited to e.g., hot flashes, night sweats, insomnia, mood swings/irritability, memory or concentration problems, vaginal dryness, heavy bleeding, fatigue, depression, hair changes, headaches, heart palpitations, sexual disinterest, urinary changes and weight gain.

Menopause may be marked by the final period or ovulation cycle of the female subject, but is generally not an abrupt event, rather a gradual process. The clearest indication of menopause is the absence of an ovulation for one year, at which point a woman may be referred to as postmenopausal. It is also possible to diagnose menopause by testing hormone levels. One important test measures the levels of follicle-stimulating hormone (FSH), which steadily increases as a woman ages.

The age of menopause onset as well as the age of menopause completion may vary. Eight out of every 100 women will stop menstruating before age 40 and five out of every 100 women will continue to have periods until they are about 60 years of age. The average age of menopause is 51 and often occurs between the ages of 45 and 55. Menopause before the age of 40 may be referred to as premature menopause. Menopause before the age of 45 may be referred to as early menopause.

Some factors may be useful in predicting the onset and/or completion of menopause including but not limited to e.g., family history, body type, and lifestyle choices. The age at which menstruation began is not necessarily a predictor of when menopause will occur. For example, a woman beginning menstruation early or having entered puberty early will not necessarily experience early menopause.

The term “premenopause” generally refers to menstrual cycling that is relatively normal for a subject. A subject in premenopause may experience some gradual change in menstruation across a lifecycle, such as alteration in cycle length, changes in period pain or other premenstrual symptoms. However, during premenopause, on average, menstrual cycling will occur at regular intervals, including about monthly. The length of menstrual cycles may vary and may range from 21 to 45 days including 21 to 35 days in women of reproductive age, where the average cycle is 28 days.

The terms “perimenopause” or “menopausal transition” may be used interchangeably and generally refer to the interval in which many woman begin to experience one or more symptoms of menopause including e.g., irregular menstrual cycles. Perimenopause generally refers to the period just before menopause.

Further definitions and information related to menopause, including pre- and post-menopause may be found in Menopause: Full Guideline. NICE Guideline, No. 23. National Collaborating Centre for Women's and Children's Health (UK). London: National Institute for Health and Care Excellence (UK); 2015 Nov. 12; the disclosure of which is incorporated herein by reference in its entirety.

Whereas infertility and/or menopause, including premature menopause and early menopause, may not be classically considered diseases per se, for simplicity reference to diseases made herein will include fertility related conditions such as infertility, menopause, premature menopause, early menopause, and the like.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as C_{H}). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction;

(4) F(ab)₂ is a dimer of two Fab′ fragments held together by two disulfide bonds;

(5) Fv, defined as a genetically engineered fragment containing the variable region of the light chain and the variable region of the heavy chain expressed as two chains;

(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and

(7) “3G”, including single domain (typically a variable heavy domain devoid of a light chain) and “miniaturized” antibody molecules (typically a full-sized Ab or mAb in which non-essential domains have been removed).

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with infertility, e.g. those having infertility) as well as those in which prevention is desired (e.g., those with increased susceptibility to infertility; those subject to premature menopause; those suspected of having infertility; etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent that increases fertility and/or compositions is an amount that is sufficient, when administered to the individual, to palliate, ameliorate, stabilize, reverse, prevent, slow or delay the progression or onset of infertility by, for example, ablating senescent cells.

### DETAILED DESCRIPTION

Methods of increasing fertility in a female subject are provided. Aspects of the methods include ablating senescent cells present in the reproductive system of the subject to increase the fertility of the female subject. Ablating senescent cells in the provided methods may include where senescent cells of the reproductive system are specifically ablated.

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present methods are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order which is logically possible.

### Methods

As summarized above, the methods of the present disclosure include increasing fertility in a female subject. Methods of the present disclosure may include specifically ablating senescent cells present in the reproductive system of a subject. In some instances, a subject may be administered an effective amount of an agent that specifically ablates senescent cells present in the reproductive system of the subject to increase fertility in the subject. In the present methods, any senescent cell present in the reproductive system may be targeted, including but not limited to e.g., senescent cells present in the ovary, senescent cells present in the fallopian tubes, senescent cells present in the uterus, and the like.

Senescent cells targeted in the subject methods may include senescent cells present in or about the ovary of the subject. In some instances, senescent cells that directly affect the functioning of the ovary of a subject may be targeted. Such cells may reside inside or outside of the ovary but generally influence female fertility through direct influences on the female ovary. Agents, discussed in more detail below, administered to a subject to ablate senescent cells may be administered systemically or locally and by various routes of administration to target senescent cells, including e.g., senescent cells of the ovary.

Senescent cells targeted in the subject methods may include senescent cells present in or about the fallopian tubes of the subject. In some instances, senescent cells that directly affect the functioning of the fallopian tubes of a subject may be targeted. Such cells may reside inside or outside of the fallopian tubes but generally influence female fertility through direct influences on the female fallopian tubes. Agents, discussed in more detail below, administered to a subject to ablate senescent cells may be administered systemically or locally and by various routes of administration to target senescent cells, including e.g., senescent cells of the fallopian tubes.

Senescent cells targeted in the subject methods may include senescent cells present in or about the uterus of the subject. In some instances, senescent cells that directly affect the functioning of the uterus of a subject may be targeted. Such cells may reside inside or outside of the uterus but generally influence female fertility through direct influences on the female uterus. Agents, discussed in more detail below, administered to a subject to ablate senescent cells may be administered systemically or locally and by various routes of administration to target senescent cells, including e.g., senescent cells of the uterus.

Senescent cells targeted for ablation in the subject methods will vary. Cellular senescence differs from quiescence and terminal differentiation in several aspects. Senescent cells have characteristic morphological changes such as enlargement, flattening, and increased granularity. Generally, senescent cells do not divide even if stimulated by mitogens. Senescence may involve activation of one or more markers of senescence and/or tumor related genes (e.g., tumor suppressors) such as p53 and/or Rb and their regulators such as p16^{INIK4a}, p21, and ARF. In many instances, senescence is irreversible. In some instances, senescence may be reversible, e.g., when p53 or Rb is inactivated. Senescent cells express increased levels of plasminogen activator inhibitor (PAI) and exhibit staining for beta-galactosidase activity at pH 6. Irreversible G1 arrest is mediated by inactivation of cyclin dependent kinase (CdK) complexes which phosphorylate Rb. P21 accumulates in aging cells and inhibits CdK4-CdK6. P16 inhibits CdK4-CdK6 and accumulates proportionally with beta-galactosidase activity and cell volume. p21 is expressed during initiation of senescence but need not necessarily persist. p16 expression helps maintain senescence once initiated.

Cellular senescence may include replicative senescence, which is related to the progressive shortening of telomeres with each cell division. Senescence is induced when certain chromosomal telomeres reach a critical length. In some instances, senescence can be abrogated by the expression of telomerase which lengthens telomeres; human fibroblasts undergo replication indefinitely when transfected to express telomerase.

Pathways to senescence other than reproductive senescence, i.e., non-reproductive senescence, include stress-induced premature senescence (SIPS). Oxidative stress can shorten telomeres and hyperoxia can induce senescence. Gamma irradiation of human fibroblasts in early to mid G1 phase causes senescence in a p53-dependent manner. Ultraviolet radiation also induces cellular senescence. Other agents that can induce cellular senescence include e.g., hydrogen peroxide, sodium butyrate, 5-azacytadine, transfection with the Ras oncogene, 5-bromodeoxyuridine, chemotherapeutic agents (e.g., doxorubicin, cisplatin, and others) and generally agents that damage DNA.

Cells may become senescent as a result of aging. Cells may also become senescent after exposure to an environmental, chemical, or biological insult or as a result of a disease. Senescent cells may exhibit any one or more of the following characteristics: senescence growth arrest that is essentially permanent and cannot be reversed by known physiological stimuli; increase in size (e.g., more than twofold relative to the size of the non-senescent counterpart); expression of a senescence-associated β-galactosidase; increase in lysosomal mass; expression of p16^{INK4a}, persistent DDR signaling, harboring of persistent nuclear foci (DNA segments with chromatin alterations reinforcing senescence (DNASCARS)); dysfunctional telomeres or telomere dysfunction induced foci (TIF); expression and/or secretion of molecules associated with senescence; loose structural nuclei proteins such as Lamin B1 or chromatin-associated proteins such as histones and HMGBI.

In some instances, senescent cells exert clinically significant paracrine effects, including when the senescent cells represent only a small portion of the cells of the organ or tissue in which they reside. For example, senescent cells may compose only 10% of less of the cells of a particular organ, including e.g., 9% or less, 8% or less, 7% or less, 6% or less, 5% or less, 4% or less, 3% or less, 2% or less or 1% or less. Accordingly, in some instances, the subject methods include eliminating senescent cells while leaving intact the majority of the cellular compartment from which the senescent cells originate. In some instances, ablating senescent cells can negate or reduce harmful paracrine effects that result from the presence of senescent cells.

Senescent cells include those that express one or more markers of senescence. As such, the described methods may include ablating senescent cells based on their expression of one or more markers of senescence. Agents described herein may target senescent cells based on their expression of one or more senescence markers.

Useful markers of cellular senescence include but are not limited to e.g., SA-beta galactosidase (SA-βgal), p16, p53, Rb, p16^{INIK4a}, p21, ARF, DEP1, NTAL, EBP50, STX4, VAMP3, ARMX3, B2MG, LANCL1, VPS26A and PLD3 and the like, as well as those described in Althubiti et al., (Cell Death and Disease (2014) 5, e1528); Baker et al. (Nature 479(7372): 232-236); Baker et al. (Nature. 2016; 530(7589): 184-189; Bennett et al. Science. 2016; 354(6311): 472-477; Jeon et al., Nat Med. 2017 doi: 10.1038/nm.4324); the disclosures of which are incorporated herein by reference in their entirety.

In some instances, senescent cells may be identified based on the cellular, tissue or organ expression of a senescent cell-associated molecule or the presence of a senescent cell-associated molecule in a particular cell, tissue or organ. Senescent cell-associated molecules include but are not limited to e.g., growth factors, proteases, cytokines (e.g., inflammatory cytokines), chemokines, cell-related metabolites, reactive oxygen species (e.g., hydrogen peroxide), and the like.

Useful senescent cell-associated molecules also include secreted factors which may make up the pro-inflammatory phenotype of a senescent cell, including e.g., GM-CSF, GROα, GROαβγ, IGFBP-7, IL-Iα, IL-6, IL-7, IL-8, MCP-1, MCP-2, MIP-Iα, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-Iβ, MCP-4, MIF, MIP-3α, MMP-12, MMP-13, MMP-14, NAP2, Oncostatin M, osteoprotegerin, PIGF, RANTES, sgp130, TIMP-2, TRAIL-R3, Acrp30, angiogenin, Ax1, bFGF, BLC, BTC, CTACK, EGFR, Fas, FGF-7, G-CSF, GDNF, HCC-4, 1-309, IFN-γ, IGFBP-1, IGFBP-3, IL-I RI, IL-11, IL-15, IL-2R-α, IL-6 R, I-TAC, Leptin, LIF, MMP-2, MSP-α, PAI-I, PAI-2, PDGFBB, SCF, SDF-1, sTNF RI, sTNF RII, Thrombopoietin, TIMP-1, tPA, uPA, uPAR, VEGF, MCP-3, IGF-1, TGF-β3, MIP-1-delta, IL-4, FGF-7, PDGF-BB, IL-16, BMP-4, MDC, MCP-4, IL-10, TIMP-1, Fit-3 Ligand, ICAM-1, Ax1, CNTF, INF-γ, EGF and BMP-6.

Useful senescent cell-associated molecules also include senescence messaging secretome (SMS) factors including but not limited to e.g., IGF1, IGF2, and IGF2R, IGFBP3, IDFBP5, IGFBP7, PAL1, TGF-β, WNT2, IL-Iα, IL-6, IL-8, and CXCR2-binding chemokines. Other useful senescent cell-associated molecules may include the gene products of the following genes: MMP1, WNT16B, SFRP2, MMP12, SPINK1, MMP10, ENPP5, EREG, BMP6, ANGPTL4, CSGALNACT, CCL26, AREG, ANGPTI, CCK, THBD, CXCL14, NOV, GAL, NPPC, FAM150B, CST1, GDNF, MUCL1, NPTX2, TMEM155, EDN1, PSG9, ADAMTS3, CD24, PPBP, CXCL3, MMP3, CST2, PSG8, PCOLCE2, PSG7, TNFSF15, C17orf67, CALCA, FGF18, IL8, BMP2, MATN3, TFP1, SERPINI 1, TNFRSF25, and IL23A.

Senescent cell-associated molecules also include senescent cell-associated proteins, which include cell surface proteins (or receptors) that are expressed on senescent cells, which include proteins that are present at a detectably lower amount or are not present on the cell surface of a non-senescent cell.

Identification of cells to be ablated in the herein described methods and/or targeting of cells to be ablated may, in some instances, be based on senescent marker expression and/or the expression or presence of one or more senescent cell-associated molecules. For example, an organ (such as the ovary, fallopian tube, uterus, etc.), a tissue (such as an ovarian tissue, a fallopian tube tissue, an uterine tissue, etc.) or a cell (such as an ovary cell type, a fallopian tube cell type, uterine cell type, etc.) may be screened for senescence, e.g., by evaluating for one or more senescent markers or senescent-associated molecules and detected senescent cells therein may be targeted for ablation.

In some instances, the methods described herein may include targeting a specific cell type for ablation, including where the specific cell type is targeted based on having a senescent phenotype and/or expression of one or more senescence markers. Specific cell types that may be targeted include cell types present in the reproductive system of the female subject.

Specific cell types that may be targeted include cell types present in the ovary. Ovary cell types include cells present in one or more ovary regions including e.g., the ovarian cortex, ovarian medulla or germinal epithelium. Specific ovary cell types that may be targeted for ablation include e.g., germinal epithelium cells, ovarian stem cells, primordial follicle cells, primary follicle cells, secondary follicle cells, vesicular follicle cells, oocytes, theca cells, granulosa cells, ovarian stromal cells and corpus luteum cells. In some instances, a cell targeted for ablation may be a specific type of ovarian cell that is senescent, e.g., expresses one or more markers of cellular senescence. In some instances, senescent ovarian cells may be targeted generally irrespective of the specific cell type of the ovarian cell. In some instances, one or more specific ovarian cell types may not be targeted, including e.g., where senescent ovarian cells are targeted in general with the exception of one or more specific ovarian cell types.

Ovary cell types may express one or more ovary lineage markers. Useful ovarian lineage markers include those expressed during development of the ovary as well as in adult ovarian tissue. Ovarian lineage markers may include e.g., germline markers (e.g., Oct4, Mvh, Dazl, Blimp1, Fragilis, Stella, Rex1, etc., including human homologs thereof), germ cell-specific markers (e.g., germ cell nuclear antigen (GCNA), cKIT, mouse vasa homolog (MVH), etc., including human homologs thereof), ovarary-specific markers whether expressed during development and/or during juvenile and/or adult stages (e.g., Oocyte-G1, Zp3 (zona pellucida sperm binding protein 3), Nobox (newborn ovary homeobox protein), and Gdf9 (growth differentiation factor 9), etc., including human homologs thereof), ovarian stroma markers (e.g., COUP-TFII/NR2F2, etc., including human homologs thereof), granulosa cell and granulosa precursor cell markers (e.g., FOXL2, leucine-rich repeat-containing G protein-coupled receptor 5 (LGR5), etc., including human homologs thereof), ovarian surface epithelium markers (e.g., lymphocyte antigen 6 complex, locus A (LY6A) etc., including human homologs thereof), stem cell markers (e.g., NANOG, secreted frizzled related protein 1 (SFRP1), LIM homeobox 9 (LHX9), ALDH1A1, ALDH1A2, etc., including where stem cell markers are expressed in the ovarian surface epithelial), oocyte-specific markers (e.g., DAZL, VASA, STELLA, ZP, GDF9B, SCP3, C-MOS, etc., including human homologs thereof), endothelial-like markers (e.g., TIE (tyrosine kinase with Ig-like and epidermal growth factor [EGF]-like domains), TEK (endothelial-specific receptor tyrosine kinase), von Willebrand factor, cKIT, CD31, FLT-1 (VEGF receptor 1), etc., including human homologs thereof), endothelial markers (e.g., CD14, CD45, CD133, VEGF-R2, etc., including human homologs thereof), granulosa-like cell markers (e.g., FOXL2, CYP19A1, FSHR, AMH (anti-Müllerian hormone), AMHR2, etc., including human homologs thereof), those markers described in Hummitzsch et al., Endocr Rev. 2015 February; 36(1): 65-91; the disclosure of which is incorporated herein by reference in its entirety.

Useful ovary lineage markers identifying ovary cell types also include but are not limited to e.g., Lgr5, FOXL2, NR2F2 and those described in e.g., Rastetter et al. (Dev Biol. 2014 Oct. 15; 394(2):242-52) and the human homologs thereof.

Specific cell types that may be targeted include cell types present in the fallopian tubes. Fallopian tube cell types include cells present in one or more fallopian tube regions including e.g., the serosa, subserosa, the lamina propria and the innermost mucosal layer. Specific fallopian tube cell types that may be targeted for ablation include e.g., ciliated cells and peg cells. In some instances, a cell targeted for ablation may be a specific type of fallopian tube cell that is senescent, e.g., expresses one or more markers of cellular senescence. In some instances, senescent fallopian tube cells may be targeted generally irrespective of the specific cell type of the fallopian tube cell. In some instances, one or more specific fallopian tube cell types may not be targeted, including e.g., where senescent fallopian tube cells are targeted in general with the exception of one or more specific fallopian tube cell types.

Fallopian tube cell types may express one or more fallopian tube cell-type markers. Useful fallopian tube cell-type markers include those expressed during development of the fallopian tubes as well as in adult fallopian tube tissue. Fallopian tube cell-type markers may include e.g., markers of ciliated cells (e.g., β-tubulin; TUBB4) or the absence thereof, markers of secretory cells (e.g., PAX8) or the absence thereof, epithelial cell adhesion molecule (EPCAM), CD44, integrin alpha-6 (ITGA6), membrane metallo-endopeptidase (MME), gynecological epithelial cell marker CA125, Von Willenbrand Factor VIII or the absence thereof, fibroblast surface protein, cytokeratin, laminin, vimentin, fibronectin, p53, MIB1, collagens (e.g., collagen I, collagen IV, etc.), MUC-1, FOXJ1, as well as those markers described in Paik et al., Stem Cells. (2012) 30(11): 2487-2497; Lawrenson et al., BMC Cell Biology (2013) 14:43; Merritt et al. PLoS One. (2013) 26; 8(11):e80314 and Ng & Barker, Nature Reviews Molecular Cell Biology (2015) 16:625-638; the disclosures of which are incorporated herein by reference in their entirety.

Specific cell types that may be targeted include cell types present in the uterus. Uterine cell types include cells present in one or more uterine regions including e.g., the endometrium, the myometrium or the perimetrium. Specific uterine cell types that may be targeted for ablation include e.g., endometrial epithelial cells, endometrial stromal cells, endometrial gland cells, smooth muscle cells, perimetrium mesothelial cells. In some instances, a cell targeted for ablation may be a specific type of uterine cell that is senescent, e.g., expresses one or more markers of cellular senescence. In some instances, senescent uterine cells may be targeted generally irrespective of the specific cell type of the uterine cell. In some instances, one or more specific uterine cell types may not be targeted, including e.g., where senescent uterine cells are targeted in general with the exception of one or more specific uterine cell types.

Uterine cell types may express one or more uterine cell-type markers. Useful uterine cell-type markers include those expressed during development of the uterus as well as in adult uterine tissue. Uterine cell-type markers may include e.g., CD10, h-caldesmon, desmin, smooth muscle actin, interferon-induced transmembrane protein 1 (IFITM1), oestrogen receptor, progesterone receptor, β₃ integrin, C-kit/CD117, CD34, bcl-2, and Ki67, as well as those markers described in Parra-Herran et al. Modern Pathology (2014) 27:569-579; Classen-Linke et al., Human Reproduction Update (1998) 4(5):539-54; Cho et al., Fertil Steril. (2004) 81(2):403-7; the disclosures of which are incorporated herein by reference in their entirety.

As will be readily understood with regard to the use of cell-type markers, the presence or absence of a particular marker may be employed to identify and/or target a specific cell type. As such, in some instances, the absence of a particular marker may be employed to identify, alone or in combination with the presence or absence of other markers, a certain cell type. In some instances, the presence of a particular marker may be employed to identify, alone or in combination with the presence or absence of other markers, a certain cell type.

In some instances, a combination of markers may be employed to target a particular senescent cell. For example, as described above, a cell may express a senescence marker and a marker of a reproductive system cell type or a reproductive cell lineage marker. Other useful combinations include combinations of multiple senescence markers, combinations of multiple ovary lineage markers, and the like. Useful markers may be expressed on the surface of a target cell, i.e., cell surface markers, or markers (e.g., proteins, nucleic acids, etc.) expressed within a cell, i.e., intracellular markers. As such single senescent reproductive system cell types or multiple senescent reproductive system cell types, including all senescent cells of the reproductive system, (or all the senescent cells of a targeted reproductive tissue, e.g., ovary, fallopian tube, uterus, etc.) may be targeted for ablation according to the methods described herein.

As summarized above, the methods of the present disclosure include increasing the fertility of a subject. Various subjects may be treated according to the subject methods. In some instances, the treated subject is a healthy subject, i.e., a subject not suffering from any chronic or acute affliction, condition or disease, including but not limited to e.g., cancer and the like. The fertility of healthy subjects may be increased, e.g., to the normal level of fertility of a corresponding but younger subject, including a subject 5 years younger, 10 years younger, 15 years younger, 20 years younger, 25 years younger, etc. In some instances, fertility of a subject may be increased to a level higher than that of a normal healthy individual of corresponding age.

Infertility, as used herein, is generally not considered a disease and, as such, a treated subject may be a healthy subject with infertility. Such subjects may be treated, e.g., to at least reduce the infertility in the subject and improve the subject's fertility, including where the subject's fertility is increased to that of a normally fertile subject of corresponding age (i.e., below average fertility may be increased to correspond with expected average fertility of a subject of corresponding age and health). In some instances, the fertility of a subject with below average fertility may be increased to a level above that of a normally fertile subject of corresponding age.

As summarized above, infertility may be the consequence of one or more adverse health conditions or disease. As such, in some instances, the methods may include treating an unhealthy subject (i.e., a subject with a disease or adverse health condition) with infertility that is a result of a disease or adverse health condition of the subject. Such subjects may be treated, e.g., to at least reduce the infertility in the subject and improve the subject's fertility, including where the subject's fertility is increased to that of a normally fertile subject of corresponding age (i.e., below average fertility may be increased to correspond with expected average fertility of a subject of corresponding age and health). In some instances, in the fertility of a subject with below average fertility may be increased to a level above that of a normally fertile subject of corresponding age.

The fertility of a subject may be determined or measured by any convenient means including e.g., the ability of the subject to conceive upon regular attempts (i.e., normally productive intercourse), the ovulation frequency of the subject, measuring the levels of one or more fertility related hormones or other fertility biomarkers, one or more imaging based diagnostic methods (including e.g., ultrasound imaging of the reproductive system including the ovaries, fallopian tubes and/or uterus), analysis and/or counting of one or more reproductive structures (e.g., ovarian structures, fallopian structures, uterine structures) or cell types observed or retrieved from a reproductive tissue of the subject (e.g., as part of an in vitro fertilization (IVF) procedure or other infertility intervention).

In some cases, the fertility of the subject may be increased to prolong fertility in the subject, including healthy and unhealthy subjects. Fertility may be prolonged beyond the average of menopause, including where the average of menopause is adjusted or calculated for a particular subject, e.g., as based on demographic, life history, family history, or other factors. Accordingly, in some instances, fertility may be prolonged 1 year or more beyond the average age of menopause onset, including e.g., 2 years or more, 3 years or more, 4 years or more, 5 years or more, 10 years or more, etc. Where a subject is expected to have premature or early menopause onset (e.g., based on particular life history, family history, or one or more risk factors) the method may increase fertility in the subject closer to a normal level, including e.g., closer to the average age of menopause onset, including e.g., within 10 years or less of the average age of menopause onset, e.g., within 5 years or less, within 4 years or less, within 3 years or less, within 2 years or less, within 1 year or less, etc. In some instances, fertility of a subject at risk of premature or early menopause may be prolonged to at least an average age of menopause onset or beyond.

As summarized above, the present disclosure includes methods of treatment for increasing fertility, restoring fertility of an infertile subject, increasing the duration of fertility in a subject at risk of premature or early menopause, etc. Such methods may include administering an effective amount of an agent to the subject. Effective amounts of the agents are those administered amounts effective to increase fertility, as described above, and/or amounts administered in order to prevent, ameliorate, inhibit the development of, or treat the fertility related symptoms of diseases or disorders related to or caused by infertility.

Treatment regimens, including the administration of an agent to a subject, of the present disclosure may vary. In some instances, the agent may be administered chronically. In some instances, the agent may be administered intermittently or for a predetermined duration or until a predetermined level of fertility is reached. Dosing of the agent may vary and may include e.g., semi-daily, daily, bi-daily, semi-weekly, weekly, bi-weekly, semi-monthly, monthly, bi-monthly, quarterly, semi-yearly, yearly, bi-yearly, etc.

As noted above, subjects treated according to the herein described methods may be treated chronically or for a finite time period. For example, the subject may be treated, e.g., administered an agent, according to a predetermined treatment schedule, the length of which may vary and may range from multiple weeks or longer, including but not limited to e.g., 2 weeks or more, 3 weeks or more, a month or more, 2 months or more, 3 months or more, 4 months or more, 5 months or more, 6 months or more, 7 months or more, 8 months or more, 9 months or more, 10 months or more, 11 months or more, a year or more, 2 years or more, 3 years or more, 4 years or more, etc. A predetermined treatment course may include a dosing schedule, e.g., one or more dosing schedules described herein, including e.g., twice daily, daily, bi-daily, weekly, bi-weekly, monthly, bi-monthly, quarterly, twice a year, yearly, bi-yearly, and the like.

In some instances, a treatment schedule may include periodic dosing of an agent to a subject, i.e., may include one or more “on” and “off” periods of dosing over the course of treatment. An “off” period of a doing schedule may be referred to as a holiday period. In a holiday period no agent for ablating senescent cells is delivered. A “holiday period”, may vary, but in representative embodiments is at least about 1 day, such as at least about 2 days, including at least about 5 days, at least about 10 days, at least about 15 days, or longer. As such, embodiments of the methods include non-chronic (i.e., non-continuous) application of the agent, e.g., non-chronic administration of an agent that targets senescent cells.

A given course of treatment may include one or more holiday periods of the same or different length, including but not limited to e.g., one holiday period or more, two holiday periods or more, three holiday periods or more, four holiday periods or more, etc.

Particular treatment schedules may be generically applied to subjects broadly or specifically tailored or designed to the needs of individual subjects or groups of related subjects.

### Ablating Senescent Cells

As summarized above, the provided methods may include increasing fertility in a subject by ablating senescent cells in the subject. Senescent cell ablation for increasing fertility of a female subject may be performed systemically or locally. Senescent cells of the ovary may be specifically targeted in the subject methods. Senescent cells of the fallopian tubes may be specifically targeted in the subject methods. Senescent cells of the uterus may be specifically targeted in the subject methods.

Senescent cell ablation may be performed through the administration of an effective amount of an agent to the subject that ablates senescent cells, including where such an agent specifically ablates senescent cells of a reproductive tissue (e.g., the ovary, the fallopian tubes, the uterus, etc.). Specific ablation of senescent cells of reproductive tissues may be achieved through a variety of approaches including but not limited to e.g., specific delivery of an agent to the reproductive tissue of the subject such that the effects of the agent occur only or at least primarily locally in the reproductive tissue, delivery of an agent that is specific to the reproductive tissue of interest or cells of the reproductive tissue of interest such that the effects of the agent occur only or at least primarily in the targeted reproductive tissue or cells of the reproductive tissue, and the like. Agents that specifically target one or more reproductive tissues of interest or cells thereof may, in some instance, be administered systemically to specifically ablate senescent reproductive tissue cells.

Useful agents include small molecule agonists, non-peptide small molecule agonists, small molecule antagonists, non-peptide small molecule antagonists, peptide agonists, peptide antagonists, interfering RNAs (e.g., siRNA, shRNA, etc.), antibodies (e.g., neutralizing antibodies, function blocking antibodies, etc.), aptamers, and the like. In some instances, the effectiveness of an agent may be confirmed using an in vitro or in vivo assay, including e.g., where the effectiveness of the agent is compared to an appropriate control or standard, e.g., a conventional therapy for a condition, a mock therapy for a condition, no treatment, etc. In some instances, agents that specifically target senescent cells may be referred to as senolytic agents.

A senolytic agent is an agent that “selectively” (preferentially or to a greater degree) destroys, kills, removes, or facilitates selective destruction of senescent cells. In other words, the senolytic agent destroys or kills a senescent cell in a biologically, clinically, and/or statistically significant manner compared with its capability to destroy or kill a non-senescent cell. A senolytic agent is used in an amount and for a time sufficient that selectively kills established senescent cells but is insufficient to kill (destroy, cause the death of) a non-senescent cell in a clinically significant or biologically significant manner. Senolytic agents include those that alter at least one signaling pathway in a manner that induces (initiates, stimulates, triggers, activates, promotes) and results in (i.e., causes, leads to) death of the senescent cell. The senolytic agent may alter, for example, either or both of a cell survival signaling pathway (e.g., Akt pathway) or an inflammatory pathway, for example, by antagonizing a protein within the cell survival and/or inflammatory pathway in a senescent cell. In some instances, senolytic agents release inhibition of apoptotic cell death. In some instances, senolytic agents induce (i.e., activate or stimulate) apoptotic cell death. In some instances, senolytic agents inhibit cell survival pathways. In some instances, senolytic agents stimulate immune or inflammatory pathways.

Useful agents include those that specifically bind senescent cells, including those that bind with higher affinity to senescent cells than non-senescent cells. Such agents may have an inherent ablative function (e.g., through a toxic effect upon binding, through recruitment of cells (e.g., immune cells) that ablate the senescent cell to which the agent binds, etc.) or an ablative function may be engineered into the agent (e.g., through attachment of a cytotoxic moiety to the agent, through attachment of an epitope to the agent to which ablative cells are directed, etc.). Agents that specifically bind senescent cells include those that bind a molecule expressed on the cell surface of a senescent cell, such as e.g., an antibody that binds a molecule expressed on the cell surface of a senescent cell, a non-peptide small molecule that binds a molecule expressed on the cell surface of a senescent cell, a peptide that binds a molecule expressed on the cell surface of a senescent cell, a ligand for a receptor expressed on the cell surface of a senescent cell, receptor for a ligand expressed on the cell surface of a senescent cell, and the like.

Useful agents include those that target senescent cell specific antigens. By targeting senescent cell specific antigens the agents can specifically affect senescent cells while minimizing off-target effects to non-target, i.e., non-senescent cells. Non-senescent cells may vary and may include e.g., proliferating cells, quiescent cells, etc., including e.g., non-senescent preadipocytes, non-senescent endothelial cells, non-senescent fibroblasts, non-senescent neurons, non-senescent epithelial cells, non-senescent mesenchymal cells, non-senescent, smooth muscle cells, non-senescent macrophages, non-senescent chondrocytes, non-senescent cells of the ovary (e.g., non-senescent cells of the ovarian cortex, non-senescent cells of the ovarian medulla, non-senescent cells of the germinal epithelium, non-senescent germinal epithelium cells, non-senescent ovarian stem cells, non-senescent primordial follicle cells, non-senescent primary follicle cells, non-senescent secondary follicle cells, non-senescent vesicular follicle cells, non-senescent oocytes, theca cells, non-senescent granulosa cells, non-senescent ovarian stromal cells, non-senescent corpus luteum cells, non-senescent cells of the fallopian tubes, non-senescent cells of the uterus, and the like).

Senescent cell-specific antigens include, e.g., Mutant beta-actin; Beta-actin (ACTB) protein; Drug resistance-related protein (LRP); Major vault protein (MVP); Thyroid hormone binding protein precursor; Prolyl4-hydroxylase, beta subunit precursor (P4HB); Chain A, human protein disulfide isomerase (PDI); Electron-transfer-flavoprotein, beta polypeptide (ETFP); ATP synthase, H+ transporting, mitochondrial F1 complex, alpha subnnit precursor; Unnamed protein product (GI: 35655); Unnamed protein product (GI: 158257194); Unnamed protein product (GI: 158259937) and Cathepsin B (CTSB) as described in U.S. Patent Application Pub. No. US 2016/0166718 A1; the disclosure of which is incorporated herein by reference in its entirety. Accordingly, agents useful in the subject method may include agents that bind to a senescent cell-specific antigen, including e.g., agents that specifically bind one of the senescent cell-specific antigens described herein. In some instances, the agent that specifically binds a senescent cell-specific antigen may be an antibody that specifically binds the senescent cell-specific antigen.

In some instances, an agent administered for ablating senescent cells may be a small molecule, including e.g., peptide and non-peptide small molecules. A “small molecule” is a low molecular weight, generally less than about 1 kDa, organic compound that may regulate a biological process or otherwise affect a living cell. Most drugs are small molecules. The size of small molecules may vary and can range from about 100 D or less to about 1 kDa or more, including but not limited to e.g., 100 D to 1 kDa, 100 D to 900 D, 100 D to 800 D, 100 D to 750 D, 100 D to 600 D, 100 D to 500 D, 200 D to 800 D, 200 D to 600 D, 300 D to 800 D, 300 D to 600 D, etc. Small molecule agents useful in particular contexts may be identified through small molecule drug screens, including in vitro and in vivo screen. Small molecules may also be rationally designed, e.g., to interfere with a known binding interaction, or be based on or derived from a larger molecule or lead compound.

Useful small molecule senolytic agents include e.g., ABT263 as described in Chang et al. (Nat Med. 2016 January; 22(1):78-83); the disclosure of which is incorporated by reference herein in its entirety.

Useful small molecules also include those selected based on their ability to specifically bind senescent cells. Non-limiting examples of useful small molecules include those that specifically bind to one or more of the senescent cell-specific antigens described herein as well as those capable of mimicking the physical, chemical, biological, and/or targeting characteristics of senescent cell-binding peptides described herein (i.e., senescent cell-binding peptide mimetics). Non-limiting examples of small molecules selected based on their ability to specifically bind senescent cells include peptide small molecules, including e.g., GVYHFAPLTPTP, SFQSHLIEFSFE, APILKLAPLIHP, GVYHFAPLTPTPGGGC, GVYHFAPLTPTPGGSKFAKFAKKFAKFAK and GVYHFAPLTPTPGGKFAKFAKKFAKFAK and peptides that are substantially identical as described in U.S. Patent Application Pub. No. US 2016/0166718 A1; the disclosure of which is incorporated herein by reference in its entirety.

Useful senolytic agents also include e.g., MDM2 inhibitors, inhibitors of one or more BCL-2 anti-apoptotic protein family members wherein the inhibitor inhibits at least BCL-xL (e.g., BCL-2/BCL-xL inhibitor; a BCL-2/BCL-xL/BCL-w inhibitor; BCL-xL selective inhibitors (e.g., benzothiazole-hydrazone compounds, aminopyridine compounds, benzimidazole compounds, tetrahydroquinolin compounds, phenoxyl compounds, etc.), Akt specific inhibitors, and the like. MDM2 inhibitors include e.g., cis-imidazoline compounds, spirooxindole compounds, benzodiazepine compounds, MDM2 inhibitors that also inhibit MDMX, Serdemetan, piperidinone compounds, and the like. Cis-imidazoline compounds include is nutlin compounds (e.g., Nutlin-3a) and dihydroimidazothiazole compounds, including e.g., those agents described in U.S. Patent Application Pub. No. US 2016/0339019 A1; the disclosure of which is incorporated herein by reference in its entirety. In some instances, useful senolytic agents include cancer therapy agents (e.g., chemotherapeutics, radiotherapeutics, etc.).

Useful agents also include those antibodies selected based on their ability to specifically bind senescent cells. Non-limiting examples of useful antibodies include those that specifically bind a senescent cell-specific antigen, including e.g., one or more of the senescent cell-specific antigens described herein. Antibodies to specific targets, e.g., senescent cell-specific antigens, may be made according to conventional methods, including e.g., immunization of a host animal will an antigen to which the produced antibody will be directed. Antibodies may be monoclonal or polyclonal.

Agents that specifically bind certain cells, e.g., senescent cells, reproductive tissue cell types (e.g., ovarian cell types, fallopian tube cell types, uterine cell types, etc.), etc., may be modified with an additional function, e.g., through the attachment of a therapeutic moiety to the antibody. As used herein, the term “therapeutic moiety” generally refers to a molecule that confers a therapeutic effect upon an agent to which it is attached or otherwise facilitates the use of a therapeutic agent to target a cell bound by the agent to which the therapeutic moiety is attached. Such therapeutic effects will vary and may include e.g., cytotoxic or cytolytic effects. In some instances, a therapeutic moiety may provide a binding site or reactive site that is bound by or otherwise acted upon by a further therapeutic agent, e.g., a drug (e.g., a small molecule drug, a therapeutic antibody, etc.), a therapeutic cell, and the like.

Therapeutic moieties may be attached to a primary agent by any convenient means, including direct or indirect covalent attachment. In some instances, an agent may be attached to a therapeutic moiety through the use of a linker moiety. By “linker moiety” is meant a sequence of amino acid residues, e.g., at least one, two, three, four, five, six, seven, eight, nine, ten, fifteen, twenty, thirty, forty, fifty, or more residues, that couples an agent (e.g., a peptide, polypeptide, protein, small molecule, antibody, or antibody fragment that target senescent cells) to, e.g., one or more therapeutic agents.

In some instances, a therapeutic moiety attached to an agent may include a cytotoxic agent. By “cytotoxic agent” is meant any naturally occurring, modified, or synthetic compound that is toxic to cells. Such agents are useful in the desired ablation of neoplastic cells, and in the treatment of other symptoms or diseases characterized by cell proliferation or a hyperactive cell population. Cytotoxic agents can also be used to target undesirable cells or tissues other than neoplastic cells or tissues, e.g., senescent cells. Cytotoxic agents include, but are not limited to, alkylating agents, antibiotics, antimetabolites, tubulin inhibitors, topoisomerase I and II inhibitors, hormonal agonists or antagonists, immunomodulators, or agents that cause cell lysis (i.e., cytolytic agents) including naturally occurring or synthetic peptides. Cytotoxic agents may be cytotoxic when activated by light or infrared (Photofrin, IR dyes, etc.), may operate through other mechanistic pathways, or be supplementary potentiating agents.

Non-limiting examples of cytotoxic agents include, e.g., antineoplastic agents such as: Acivicin; Aclarubicin; Acodazole Hydrochloride; Acronine; Adozelesin; Adriamycin; Aldesleukin; Altretamine; Ambomycin; A. metantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Camptothecin; Caracemide; Carbetimer; Carboplatin; Carmustine; Carubicin Hydrochloride; Carzelesin; Cedefingol; Chlorambucil; Cirolemycin; Cisplatin; Cladribine; CombretestatinA-4; Crisnatol Mesylate; Cyclophosphamide; Cytarabine; Dacarbazine; DACA (N-[2-(Dimethyl-amino) ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Dolasatins; Doxorubicin; Doxorubicin Hydrochloride; Droloxifene; Droloxifene Citrate; Dromostanolone Propionate; Duazomycin; Edatrexate; Eflomithine Hydrochloride; Ellipticine; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; Gemcitabine; Gemcitabine Hydrochloride; Gold Au 198; Homocamptothecin; Hydroxyurea; Idarubicin Hydrochloride; Ifosfamide; Ilmofosine; Interferon Alfa-2a; Interferon Alfa-2b; Interferon Alfa-nI; Interferon Alfa-n3; Interferon Beta-Iα; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; PeploycinSulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Rhizoxin; Rhizoxin D; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′ Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G 129R; CEP-7 51; linomide; sulfur mustard; nitrogen mustard (mechlorethamine); cyclophosphamide; melphalan; chlorambucil; ifosfamide; busulfan; N-methyl-Nnitrosourea (MNU); N,N′-Bis(2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; CI-973; DWA 2114R; JM216; JM335; Bis (platinum); tomudex; azacitidine; cytarabine; gemcitabine; 6-Mercaptopurine; 6-Thioguanine; Hypoxanthine; teniposide 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); 2-chlorodeoxyadenosine (2-Cda) and the like.

Non-limiting examples of cytotoxic agents also include lytic peptides, such as e.g., streptolysin O; stoichactis toxin; phallolysin; staphylococcus alpha toxin; holothurin A; digitonin; melittin; lysolecithin; cardiotoxin; cerebratulus A toxin, and the like.

Non-limiting examples of cytotoxic agents also include agents that induce complement-mediated cell lysis such as, for example, the immunoglobulin Fc subunit. Agents of the methods can also be coupled to any member of the phospholipase family of enzymes, including phospholipase A, phospholipase B, phospholipase C, phospholipase D, a catalytically active subunit thereof, and the like.

As summarized above, a therapeutic moiety may provide a binding site or reactive site that is bound by or otherwise acted upon by a further therapeutic agent. As such, in some instances, a therapeutic moiety may include an epitope that is bound by a specific binding partner. Such epitopes may be specifically bound by a therapeutic agent (e.g., a drug, an antibody, a therapeutic cell, etc.) that produces or facilitates the ablation of the senescent cell. As one example, an epitope may be specifically bound by a polypeptide expressed on the surface of a therapeutic cell (e.g., an engineered immune cell) targeting the senescent cell. As another example, an epitope may be specifically bound by an antibody or other engineered immune molecule that induces or facilitates ablation of the senescent cell.

Methods of targeting an immune cell to a particular antigen or epitope (e.g., an antigen naturally expressed on the surface of a senescent cell or an epitope linked to an agent that binds senescent cells) include but are not limited to e.g., adoptive immune therapies, such as e.g., those involving chimeric antigen receptors, engineered T cell receptors, and the like. Such methods, conventionally applied for targeting antigens expressed on cancer cells, may be readily adapted for targeting antigens expressed on senescent cells, such as senescent cells of a reproductive tissue (e.g., senescent cells of the ovary, senescent cells of the fallopian tubes, senescent cells of the uterus, etc.).

Useful epitopes for targeting include but are not limited to e.g., epitope tags. Epitope tags are short amino acid sequences, e.g., 5-20 amino acid residues in length, that can be incorporated into an agent to allow for targeting of the agent once expressed in a cell, secreted from the cell, or bound to a target cell (e.g., a senescent cell). An agent that incorporates an epitope tag can be targeted by virtue of its interaction with an antibody, antibody fragment, or other binding molecule specific for the epitope tag. Nucleotide sequences encoding the epitope tag are produced either by cloning appropriate portions of natural genes or by synthesizing a polynucleotide that encodes the epitope tag. An antibody, antibody fragment, or other binding molecule that binds an epitope tag can directly incorporate its own therapeutic moiety (e.g., a cytotoxic agent) or serve as a target for a secondary antibody, antibody fragment, or other binding molecule that incorporates such a therapeutic moiety. Exemplary epitope tags that can be used as a detectable label include c-myc, hemagglutinin, and histidine tag, and the like. Furthermore, fluorescent (e.g., GFP) and bioluminescent molecules, discussed above, can also serve as epitope tags, as antibodies, antibody fragments, and other binding molecules are commercially available for binding these moieties.

As summarized above, in some instances, agents of the herein described methods may include nucleic acid agents. Useful nucleic acids include interfering nucleic acid (e.g., interfering RNAs such as siRNA, shRNA, and the like) and nucleic acids that encode for proteins and/or peptides. In some instances, an interfering nucleic acid may be employed that, when present in a senescent cell, induces death of the senescent cell. Useful targets of interfering nucleic acids include e.g., anti-apoptotic protein encoding nucleic acids, nucleic acids encoding proteins necessary for maintaining cellular metabolism, proteins necessary for maintaining senescence, and the like. Useful nucleic acid agents also include those encoding therapeutic molecules such as cytotoxic proteins or peptides.

Nucleic acid agents can be delivered directly to provide a therapeutic effect, such as interfering RNAs, or may be contained as part of a larger construct (such as an expression cassette or vector) requiring expression to deliver the therapeutic effect (such as expressed interfering nucleic acids, nucleic acids encoding therapeutic proteins or peptides, etc.). In some instances, when presented as part of an expression construct, a nucleic acid agent may be operably linked to a transcription regulatory sequence (i.e., a regulatory control sequence, such as e.g., a promoter) such that the nucleic acid agent is expressed upon activation of the promoter. In some instances, transcription regulatory sequences active in senescent cells or transcription regulatory sequences responsive to proteins expressed in senescent cells may be employed. For example, a promoter responsive to a protein expressed in a senescent cell (e.g., a senescence-responsive promoter) may be operably linked to a nucleic acid sequence encoding a cytotoxic peptide, such that upon introduction into the senescent cell the promoter is activated by the senescent cell protein and the cytotoxic peptide is expressed. In some instances, the use of senescent cell-specific transcription regulatory sequences increases the specificity of a nucleic acid agent.

In some instances, a nucleic acid agent (e.g., polynucleotide or oligonucleotide (e.g., including a shRNA)) may be delivered by a recombinant vector in which the polynucleotide or oligonucleotide of interest has been incorporated. In other embodiments, the recombinant viral vector may be a recombinant expression vector into which a polynucleotide sequence that encodes an agent (e.g., an antibody, an antigen-binding fragment, polypeptide or peptide, etc.) that inhibits a protein, including the proteins described herein, is inserted such that the encoding sequence is operatively linked with one or more regulatory control sequences to drive expression of the agent (e.g., the polypeptide, antibody, an antigen-binding fragment, or peptide, etc.). The recombinant vector or the recombinant expression vector may be a viral recombinant vector or a viral recombinant expression vector. Exemplary viral vectors include, without limitation, a lentiviral vector genome, poxvirus vector genome, vaccinia virus vector genome, adenovirus vector genome, adenovirus-associated virus vector genome, herpes virus vector genome, and alpha virus vector genome. Viral vectors may be live, attenuated, replication conditional or replication deficient, and is often a non-pathogenic (defective), replication competent viral vector.

In some instances, ablation of senescent cells may make use of the introduction of a transgene that facilitates ablation of the senescent cells. In some instances, nucleic acid agents may employ one or more components of a senesce-specific transgene. Examples of senesce-specific transgenes include those described in Baker et al. (Nature 479(7372): 232-236); Baker et al. (Nature. 2016; 530(7589): 184-189); and Jeon et al. (Nat Med. 2017 doi: 10.1038/nm.4324); the disclosures of which are incorporated herein by reference in their entirety.

In some instances, an agent employed in ablating a senescent cell may be inducible. For example, an agent may be one component of a complex that requires the presence of a drug to become active, such that the presence of the drug induces the activity of the agent. In one embodiment, an agent may be polypeptide that dimerizes (e.g., homodimerizes or heterodimerizes) in the presence of a small molecule dimerizing agent to become active (e.g., and induce cell death), including but not limited to e.g., the induced dimerization of caspase-based polypeptide agents as described in e.g., U.S. Patent Application Pub. No.: US 2011/0286980 A1, the disclosure of which is incorporated herein by reference. In some instances, where nucleic acid agents are employed, an activity (e.g., a cytotoxic activity) of the nucleic acid agent may be induced by the administration of a drug that activates expression of the nucleic acid agent, e.g., through the use of an inducible regulatory sequence, such as a promoter.

### Combination Methods

As summarized above, the methods of the present disclosure may include administering a single agent or a combination of agents, or therapeutic methods, directed to increasing the fertility of a subject. Accordingly, in some instances, the methods described include the sole use or sole administration of any of the agents described herein, whether described individually or as part of a combination therapy. In addition, any combination of any agents or therapies described herein may be employed in the subject methods.

In some instances, a single (i.e., only, sole) agent is administered to the subject for increasing fertility in the subject. In some instances, administration of a single agent may be sufficient and clinically beneficial to increase fertility. In some instances, an agent is administered as a monotherapy and is the single (i.e., only, sole) active agent administered to the subject for treating the condition. Medications that are not necessarily excluded from administration to the subject when an agent is administered as a monotherapy include, by way of non-limiting examples, medications for other purposes such as palliative care or comfort (e.g., aspirin, acetaminophen, ibuprofen, or prescription pain-killers; anti-itching topical medications) or for treating a different disease or condition, especially if the other medications are not agents for increasing fertility.

In some instances, an agent may be administered in combination with one or more additional agents. The individual agents of a combination therapy may be administered in series or may be administered simultaneously, including e.g., where the agents are combined in a cocktail. In some instances, a combination therapy (e.g., a combination of agents) agent is administered to the subject for increasing fertility in the subject. In some instances, administration of a combination therapy (e.g., a combination of agents) may be sufficient and clinically beneficial to increase fertility. In some instances, a combination therapy (e.g., a combination of agents) is administered as a cocktail and the agents of the cocktail are the only (i.e., sole) active agents administered to the subject for treating the condition.

As summarized above, in some instances, administration of an amount of an agent effective in increase fertility of a subject may be combined with one or more additional therapies, including e.g., additional therapies directed at increasing the fertility of the subject. In some instances, a therapeutic regimen for increasing the fertility of a subject may include administering platelet rich plasma to the subject. In some instances, a therapeutic regimen for increasing the fertility of a subject may include administering a small molecule inhibitor of one or more of mTOR, NFKB, and PI3-k, pathways to the subject.

### Analysis and Monitoring

As summarized above, in some instances, the methods of the present disclosure may include analyzing a subject and/or monitoring a subject, including e.g., where such analyzing and/or monitoring is employed as part of a therapeutic regimen to increase fertility. A subject may be analyzed or monitored to assess the subject's fertility, including before, during (i.e., concurrent), or following (including immediate-, short- or long-term follow-up) treatment. The fertility of a subject may be determined or measured by any convenient means including e.g., the ability of the subject to conceive upon regular attempts (i.e., normally productive intercourse), the ovulation frequency of the subject, measuring the levels of one or more fertility related hormones or other fertility biomarkers, one or more imaging based diagnostic methods (including e.g., ultrasound imaging of the reproductive system including the ovaries, fallopian tubes, uterus, etc.), analysis and/or counting of one or more reproductive structures (e.g., ovarian structures, fallopian structures, uterine structures, etc.) or cell types observed or retrieved from the reproductive system of the subject (including e.g., the ovaries, the fallopian tubes, the uterus, etc.) e.g., as part of an in vitro fertilization (IVF) procedure or other infertility intervention.

The effectiveness of one or more agents for increasing fertility in a subject and monitoring of a subject who receives one or more of the subject agents can readily be determined by a person skilled in the medical and clinical arts. One or any combination of diagnostic methods, including physical examination, medical history and performance of analytical tests and methods described herein and practiced in the art (e.g., medical imaging including e.g., ultrasound) may be employed.

In some instances, monitoring may be employed to modulate a subject's therapy. For example, in some instances, an analysis of a subject may be performed (e.g., an analysis of a subject's fertility, an analysis of the presence of senescent cells in the subject, etc.) and the treatment of the subject may be altered based on the outcome of the assessment. Such monitoring may be performed continuously of discontinuously (i.e., intermittently). As such, the frequency of monitoring may vary and may include e.g., semi-daily, daily, bi-daily, semi-weekly, weekly, bi-weekly, semi-monthly, monthly, bi-monthly, quarterly, semi-yearly, yearly, bi-yearly, etc. In some instances, the result of an assessment directed to measuring fertility of a subject may indicate whether treatment should be initiated, altered, or terminated.

Monitoring and/or analysis may be performed on the subject (i.e., in vivo) or may be performed on a sample collected from the subject (i.e., ex vivo). In some instances, subjects may be monitored or analyzed for senescence, including the presence or amount of senescence in one or both ovaries of the subject, one or both fallopian tubes of the subject, the uterus of the subject, etc. Such monitoring and/or analysis may be performed with or independent of treatment, including before, during (i.e., concurrent), or following (including immediate-, short- or long-term follow-up) treatment. In some instances, a subject may be assessed to determine if senescent cells are present in the subject's reproductive system (e.g., ovary, fallopian tubes, uterus, etc.). In some instances, a subject may be assessed to determine the amount of senescent cells present. In some instances, the result of an assessment directed to measuring or detecting senescent cells in a reproductive tissue may indicate whether treatment should be initiated, altered, or terminated. Various means of assessing senescence may be employed, including e.g., detection of one or more senescence markers, e.g., as described above, detection of one or more senescence associated molecules, e.g., as described above, or a cellular proliferation assessment, including but not limited to e.g., measuring tritiated thymidine incorporation.

The assessments and monitoring described herein may be combined with regular health assessments of a subject, including e.g., regular check-ups or diagnostic procedures, such as e.g., regular gynecological procedures. In some instances, the assessments and monitoring described herein may be performed independently from any regular health assessment of the subject.

### Pharmaceutical Compositions

Compositions (e.g., those including one or more agents described for use in the subject methods) of this disclosure can be supplied in the form of a pharmaceutical composition. Any suitable pharmaceutical composition may be employed, described in more detail below. As such, in some instances, methods of the present disclosure may include administering an inhibitor in a composition comprising an excipient (e.g., an isotonic excipient) prepared under sufficiently sterile conditions for administration to a mammal, e.g., a human.

Administration of an inhibitor to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Formulations for these dosage forms are described herein.

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a subject compound or pharmaceutical composition can be determined by one of skill in the art, with a goal of achieving local (e.g., tissue) concentrations that are at least as high as the IC50 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, Md. 20857; (available at www(dot)fda(dot)gov/cder/guidance/index(dot)htm, the disclosure of which is incorporated herein by reference).

Conversion of Animal Doses to Human Equivalent
Doses Based on Body Surface Area

| | | To Convert Animal Dose in mg/ | |
|---|---|---|---|
| | To Convert Animal | kg to HEDa in mg/kg, Either: | |
| | Dose in mg/kg to | Divide | Multiply |
| | Dose in mg/m², | Animal | Animal |
| Species | Multiply by km | Dose By | Dose By |
| Human | 37 | — | — |
| Child (20 kg)b | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeysc | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

aAssumes 60 kg human. For species not listed or for weights outside the standard ranges, HED can be calculated from the following formula: HED = animal dose in mg/kg × (animal weight in kg/human weight in kg)0.33.
bThis km value is provided for reference only since healthy children will rarely be volunteers for phase 1 trials.
cFor example, cynomolgus, rhesus, and stumptail.

A pharmaceutical composition comprising a subject compound (e.g., a senescent cell targeting agent) may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

A subject compound may be administered to the host using any convenient means capable of resulting in the desired reduction in disease condition or symptom. Thus, a subject compound can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the tissue to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a subject compound can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

A subject compound can be utilized in aerosol formulation to be administered via inhalation. A subject compound can be formulated into pressurized acceptable propellants such as dichlorodifluoromethane, propane, nitrogen and the like.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are solidified at room temperature.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of increasing fertility in a female subject, the method comprising administering to the subject an effective amount of an agent that specifically ablates senescent cells present in the reproductive system of the subject.

2. The method according to claim 1, wherein the senescent cells express a marker of cellular senescence.

3. The method according to claim 2, wherein the marker of cellular senescence is a cell surface marker.

4. The method according to claim 1, wherein the senescent cells express a marker of a reproductive tissue.

5. The method according to claim 4, wherein the marker of the reproductive tissue is selected from the group consisting of: an ovary cell lineage marker, a fallopian tube cell marker and a uterine tissue cell marker.

6. The method according to claim 4, wherein the marker is a cell surface marker.

7. The method according to claim 1, wherein the senescent cells are cells of the ovary.

8. The method according to claim 7, wherein the cells of the ovary are cell of the ovarian cortex, ovarian medulla or germinal epithelium.

9. The method according to claim 7, wherein the senescent cells are selected from the group consisting of: germinal epithelium cells, ovarian stem cells, primordial follicle cells, primary follicle cells, secondary follicle cells, vesicular follicle cells, oocytes, theca cells, granulosa cells, ovarian stromal cells, corpus luteum cells, and combinations thereof.

10. The method according to claim 1, wherein the senescent cells are cells of the fallopian tubes.

11. The method according to claim 10, wherein the cells of the fallopian tubes are cells of the serosa, subserosa, lamina propria or innermost mucosal layer.

12. The method according to claim 10, wherein the senescent cells are selected from the group consisting of: ciliated cells, peg cells, and combinations thereof.

13. The method according to claim 1, wherein the senescent cells are cells of the uterus.

14. The method according to claim 13, wherein the cells of the uterus are cells of the endometrium, myometrium or perimetrium.

15. The method according to claim 13, wherein the senescent cells are selected from the group consisting of: endometrial epithelial cells, endometrial stromal cells, endometrial gland cells, smooth muscle cells, perimetrium mesothelial cells, and combinations thereof.

16. The method according to claim 1, wherein the subject is a healthy subject.

17. The method according to claim 16, wherein the amount is effective to prolong fertility in the healthy subject beyond an average age of menopause onset.

18. The method according to claim 1, wherein the subject has below average fertility.

19. The method according to claim 18, wherein the amount is effective to at least increase fertility in the subject closer to a normal level.

20-40. (canceled)

41. The method according to claim 1, wherein the method further comprises evaluating the fertility of the female subject.
